(19) United States
(12) Patent Application Publication
(10) Pub. No.: US 2006/0149180 A1
(43) Pub. Date: Jul. 6, 2006

(54) Low-profile, radial nerve splint with interchangeable resilient digit extensor elements

(76) Inventor: Carolyn Phelen, Del Mar, CA (US)

Correspondence Address: ERIC HANSCOM, 7395 PORTAGE WAY, CARLSBAD, CA, 92011, US

(21) Appl. No.: 11/145,777

(22) Filed: Jun. 6, 2005

Related U.S. Application Data
(60) Provisional application No. 60/641,205, filed on Jan. 3, 2005.

(51) Int. Cl. A61F 5/00

(52) U.S. Cl. 602/20; 602/5; 602/21; 602/22

(57) ABSTRACT

The invention is directed toward a low-profile, simple, orthotic glove with wrist support attachment for treating loss or impairment of extensor and/or flexor muscle function in the upper extremities, particularly in the hands and fingers, due to a peripheral neuropathy. The glove is attractive, comfortable, easy to use, inexpensive to produce, and can be used with or without the wrist support attachment. The glove has channels or tunnels into which interchangeable resilient digit extensor elements, or stays, of varying resiliency are placed such that stays of greater or lesser resiliency can be inserted if the degree of the patient's extensor muscle control changes, or the medical practitioner in charge decides that there is a need to change the amount of resiliency in the resilient digit extensor elements and exercise treatment regimens.

### CROSS REFERENCE TO RELATED APPLICATIONS

None.

### STATEMENT REGARDING FEDERALLY SPONSORED RESEARCH OR DEVELOPMENT

This invention was not federally sponsored.

Inventor: Carolyn Phelen

### FIELD OF THE INVENTION

This invention relates to medical devices that compensate for functional losses in patient mobility, particularly losses associated with radial or peripheral nerve injuries. More specifically, this invention is directed toward a low-profile, simple, orthotic glove with wrist support attachment for treating loss or impairment of extensor and/or flexor muscle function in the upper extremities, particularly in the hands and fingers, due to a peripheral neuropathy. The glove is attractive, comfortable, easy to use, inexpensive to produce, and can be used with or without the wrist support attachment. The glove has channels or tunnels into which interchangeable resilient digit extensor elements, or stays, of varying resiliency are placed such that stays of greater or lesser resiliency can be inserted if the degree of the patient's extensor muscle control changes, or the medical practitioner in charge decides that there is a need to change the support and exercise treatment regimens.

### BACKGROUND OF THE INVENTION

Medical splints have been widely used for many centuries in treating a wide range of medical disorders. There exist several prehistoric skeletons which show broken limbs which had been properly set with splints such that the owner of the limb was able to live a decade or so after breaking the bone before he/she died. The earliest written and pictorial records of splints come from ancient Egypt, where splints were made from reeds, bamboo, and bark padded with linen, and used primarily for treating fractures. The next recorded innovation in splints and splinting technique occurred around 1500 BC when copper splints were used to treat burns. Around 460 BC, no less than the famous Greek medical giant Hippocrates invented, or perhaps was the first to write about, the use of leather straps to hold splints on a fracture. Indeed, Hippocrates' leather straps were the state of the art until relatively recently, when plastic was invented. In North and South America, the historical record of medicinal advancements is sparse compared with the great written histories of Egypt, Greece and Rome, but it is well documented that the use of splints to treat broken bones was a well known and advanced art in many parts of the pre-Columbian Americas at the time of initial contact with Europeans.

During the 1900's, in addition to the discovery of plastic and its multitudinous variety of uses, the medical fight against polio and the necessity of splinting a variety of injuries under less than idea battlefield conditions during the two world wars led to rapid advancement in splinting technology. Today, splints are used to deal with an incredibly wide range of medical problems and disorders, a far cry from the splint's initial use for broken bones. Indeed, splinting has its own classification system and medically-directed standards can be found for treating different situations.

One situation in which splints are used for purposes other than mending broken bones is in the treatment of peripheral neuropathies, a term which encompasses problems with the nerves outside of the brain and spinal cord, specifically the arms and legs. For example, one well known peripheral neuropathy is Guillain-Barre' syndrome, a disease having an incidence rate of approximately 1.7 cases per 100,000 annually, which arises from complications associated with viral illnesses, such as those caused by cytomegalovirus (CMV). Epstein-Barr virus (EBV), and human immunodeficiency virus (HIV), or bacterial infection, including those caused by campylobacter jejuni and Borrelia burgdorferi (Lyme disease).

Other causes of peripheral neuropathies include chronic alcoholism, infection by the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, and some inherited neurological disorders. Such diseases may affect the peripheral nervous system (PNS) and the central nervous system (CNS), as well as other body tissues. Peripheral neuropathy may involve damage to a single nerve or nerve group (mononeuropathy) or may involve multiple nerves (polyncuropathy).

Before WWII, nerves were believed to be cords and consequently received little attention. Nerve repair consisted of simple reapproximation and suturing. During WW I, nerve injuries were repaired under tension and risked disruption after repair because of extensive soft tissue injuries and significant infections. During WW II, reoccurrence of these war injuries influenced experimental studies to further investigate the anatomy of the peripheral nerve. Poor outcomes of peripheral nerve damage repair were recognized to be the result of failed axonal regeneration at the site of the repair (Colohan, 1996). An important quality of the peripheral nervous system, as compared to the central nervous system, is its remarkable ability to recover after an injury through remyelination and regeneration of the axon (Grant, 1999).

Nerves can also be affected by injury from mechanical, thermal, chemical or compression means, causing ischemia. The prognosis for recovery from these peripheral nerve injuries depends upon which structures of the nerve were damaged (i.e., axon, endoneurium, perineurim, epineurium) and how much of the nerve was damaged. (Malick, 1984; Kasch, 1984)

Classification of nerve injury is based on damage sustained by the nerve components, nerve functionality, and the ability for spontaneous recovery (Grant, 1999; Greenfield, 1997; Ristic, 2000). Seddon (1943) published his three classification of nerve injuries, and Sunderland (1951) expanded his grading system to five (Ristic, 2000).

Neuropathy involves damage to the axon of the nerve cells wherein degeneration of the axon or its surrounding myelin sheath slows or blocks nerve signal conduction at the point of the degeneration. Demyelination (destruction of the myelin sheath around the nerve cell) greatly decreases the speed of impulse conduction through the nerve. Injury mechanisms can include mechanical injury, compartment syndromes, trauma to peripheral nerves, fractures, stretch injuries and neuropathies. Neuropathies can also be caused by the effects of age, an autoimmune disorder, and other chronic diseases, including multiple sclerosis. Neuropathies can further arise due to acute injury to the nerves, such as by severing or blunt force trauma, as can occur in laceration and compression injuries.

The result of damage to the nerve fibers includes the impairment of voluntary movement or function of the area of muscle controlled by the nerve because impulses to the area are blocked. Impaired nerve stimulation to a muscle can result in weakness, decreased movement, loss of control of movement, etc. These effects, in turn, can lead to structural changes in muscle, bone, skin, hair, nails, and body organs due to reduced use of the affected area, immobility, lack of weight bearing, etc. For example, nerve injury can result in muscle weakness, atrophy, and loss of muscle mass. Injury also often results in the loss of sensation and control of the muscles served by the damaged nerves. Such damage can also give rise to infection or structural damage. Changes may include ulcer formation, poor healing, loss of tissue mass, scaring, and deformity. No matter what the cause or type of the neuropathy, the resulting situation is that due to damage to the nerve, a certain part of the body either no longer functions or functions with lesser ability than it originally functioned. The current invention serves to treat these types of problems in a unique and functional manner.

Neuropathies can be either permanent or treatable. Treatment is possible primarily because in some cases where immediate repair or re-growth is not possible, other nerves lying near to those that have been damaged may branch out and connect to the muscles that were previously served by the damaged nerves. The prognosis and speed of peripheral nerve recovery are directly dependent upon such factors as the level and severity of injury, surgical intervention, and the subsequent rehabilitative process. Severe injuries may take months or years for the affected peripheral nerve(s) to recover, if ever. In many cases, it is at least beneficial and in some cases essential that these nerve and muscle group be partially or fully immobilized, or positioned such that they can function with less stress put on the injured part of the body.

In neuropathies affecting muscles of the hand and fingers, prolonged muscle imbalance due to loss of muscle control can result in joint contractures and over-stretching or extension of denervated muscles. Without proper care, hand function recovery may be limited or may not occur at all. Though the recovery of muscle strength may occur, the loss of sensation (for example, to temperature, pain, or pressure) may not.

The principles behind splinting and caring for a neuropathy are simple and straightforward:

1. Protect denervated muscles from being overstretched. Muscles generally work in a competitive tandem, where one set of muscles plays against another set in moving an appendage one way or the other. When one set of the muscles is weakened due to injury or lack or impairment of neural function, the “regular” set of muscles can easily overpower and overstretch the weakened or impaired muscles, potentially resulting in further injury and delayed or even reduced recovery possibilities.

2. Prevent undesirable substitution patterns and establish normal hand functions. Without a splint, a patient may adapt to the imbalance and try to maintain the previously easy hand motions by using new and anatomically damaging methods.

3. Prevent joint contractures. Should a patient be allowed to substitute the aforementioned undesirable hand patterns to compensate for the loss of normal hand motion, it is highly likely that the joint will develop a contracture from being taken through less than its full range of motion.

4. Encourage patient compliance. Even the greatest medical strategies for treating peripheral nerve damage will prove useless if the patient doesn't use the splint. Among the important factors in whether a patient will actually use the splint outside of the medical practioner's line of sight include the attractiveness and appearance of the splint, along with its comfort and ease of use. For example, a number of studies, including Hannah/Hudak (2001), Alsancak (2001) and Ven Lede (2002), show that the most important factor in whether splint compliance was adhered to by patients was aesthetic appearance, with lower profile, functional use, and less structure also being important factors.

Use of a splint is typically employed during the nerve regeneration period of recovery. Splinting helps to minimize the occurrence of deforming joint contractures caused either by hyper-flexing or by hyperextension of the muscle groups in the affected muscles. Proper splinting also encourages safe and protected use of the injured hand in daily activities. This is true particularly when dealing with hand injuries, as peripheral neuropathies relating to the hand frequently require protected regular exercise to have a reasonable chance of partial or total recovery. Thus, there has existed a need for a functional splint that allows for this protected exercise and movement of the hand with patients who have suffered a peripheral neuropathy.

The invention is particularly directed toward treating patients with radial nerve peripheral nerve damage which allows them to flex their fingers but not extend them, a condition generally known as radial nerve palsy. With this condition, a patient whose hand is open can, for example, grasp a can to pick it up, but cannot unclasp his/her fingers or thumb from the can to release it. The invention allows the patient to unclasp his/her fingers from the can, place the can in its desired location, and then be ready to close the hand on another object, as the stays pull the fingers and thumb back into their original, open position.

In trying to come up with a splint which will assist patients with peripheral and radial nerve damage, it cannot be understated how important the appearance of the splint is in terms of whether the patient will actually use it. A number of studies show that the most important factor in whether a patient will use a particular splint is not how well it works or how comfortable it is, but rather, how it looks. While many in the medical field may find this fact to be disappointing, it remains a fact that cannot be discounted when assessing whether a particular product will help the patient—the best splint in the world is useless if the patient does not use it. As will be seen from some of the prior art, many of the previous attempts to treat peripheral nerve damage were highly expensive and obtrusive machines with many metal wires, springs and clamps which looked more appropriate in a horror movie than on a person. Thus, it is easy to see why many examples of the prior art were not worn substantially outside of the medical practitioner's office.

The prior art has several examples of attempts to resolve this problem Splints are obviously well know to the medical arts, having been used centuries before the first patent office was formed. The prior art also provides numerous examples of current splints for supporting or treating nerve damage-based injury to voluntary muscle mobility of the hand and digits include supports and splints for use on the hand and digits, however none of these provides a device which can treat a peripheral neurological injury to the hands and/or fingers which is both functional and attractive, comfortable, and user-friendly—in short, a splint that a patient is likely to wear. The prior art also lacks a splint which is adaptable to a range of different sized hands, is relatively inexpensive to produce, and allows the medical practitioner a wide degree of flexibility in treating the neuropathy.

The prior art includes a number of splints which allow for retention and/or strengthening of the fingers. General purpose hand splints, such as those illustrated by U.S. Pat. No. 3,938,509 to Barber and U.S. Pat. No. 5,466,202 to Stem show the range of splints—from aluminum/foam to inflatable vinyl sheets. These splints, however, do not allow for the selective treatment of peripheral and radial nerve damage as does the current invention. Some, such as U.S. Pat. No. 6,093,162 to Farleigh and U.S. Pat. No. 4,619,250 to Hasegawa, are so unwieldy as to be suitable solely for a medical office, as they require such complex machinery to work that they could not be used at a patient's home or while a patient was walking, driving, or performing other normal daily activities. A number of exercising splints, such as U.S. Pat. No. 5,113,849 to Kulken, U.S. Pat. No. 6,547,752 B2 to Holland, and U.S. Pat. No. 6,059,694 to Villepigue, teach devices used to exercise some combination of the fingers, hand, and thumb muscles and nerves. These patents, however, are unwieldy and are useful mainly for exercising, as opposed to the current invention whose uniqueness comes in large part from its ability to be worn throughout the day and to assist the user in not only exercising and improving the damaged nerves and recovering muscles, but also allowing the user to perform daily functions such as picking up a glass of water to drink, that would be otherwise impossible for the user of the invention to accomplish due to his/her neuropathy. There are also splints which serve to retain or partially retain and immobilize the user's fingers, as seen in U.S. Pat. No. 5,921,945 to Gray, and U.S. Pat. No. 5,766,142 to Hess. These splints, however, serve only to restrain the fingers rather than encouraging the damaged nerves and muscles to heal, and neither of these splints would allow for a user to clasp and unclasp his/her hand in performing normal hand functions.

There are also a number of unpatented splints on the market today which attempt to address the need for comfortable and functional splint that can be used effectively to treat patients with peripheral nerve damage. For example, there are a number of splints by Oppenheimer and Thomas which use a combination of attachment mechanisms, springs, and wires which provide a patient with a means to unbend the fingers and thumbs after the patient has bent them. These devices, however, as the pictures in their advertisements will make readily apparent, have a large number of delicate springs and wires extending up, out, down and to the side from the patient's wrist and hand up to several inches, thereby both increasing the danger that the splint will be damaged during normal use and restricting the potential uses the patient can make of his/her hand while wearing such a device. These splints are also extremely unattractive, thereby severely decreasing the likelihood that they will be used regularly by the patient.

Finally, the prior art also has several examples of glove-type splints. U.S. Pat. No. 5,014,689 to Meunchen teaches a hand brace with dorsal and palmar hand sections which attach to one another forming a fingerless glove which serves to support the wrist and hand to prevent, reduce, or control carpal tunnel syndrome. This patent does not, however, teach any type of device which relates to the fingers of the hand. Another patent relating to gloves is U.S. Pat. No. 6,010,473 to Robinson. This glove is used by an individual with nerve damage which has hampered the use of the hand, and serves to hold one or more fingers in a set, desired position, with a stated purpose of “provid[ing] comfort to the user while also serving to lessen the obviousness of any hand or finger grotesqueness.” As such, the Robinson patent does not supply a means by which a person with nerve damage to his/her hand can use a glove-like device to improve the functioning of his/her hand.

Commercially available glove-type splints include the Robinson Peripheral Nerve Splint, currently sold by AliMed Inc., is a set of two items, where the inner item is a strip of plastic running over the patient's dorsal hand, from which five “InRigger springs” project, one each to lie dorsally over each finger and the thumb. The “InRigger springs” are metal strips that are resilient enough to “pull” a finger back up after it has been flexed forward. Over this device is fitted a glove, which serves to pull the fingers back up when the metal strips exert their pull. While the Robinson splint avoids the unwieldy and unattractive springs and wires of the Oppenheimer and Thomas splints, it does not provide a complete solution to the problem of providing a simple yet effective method of allowing for flexibility in the treatment of peripheral neuropathies. Since each “InRigger” spring is made with a pre-set tensile strength, there is no adjustability possible. It is also fairly cumbersome to use, as the user first has to align the inner part and then pull over the glove portion, an activity not easy for someone with limited, at best, use of one hand. Finally, although more attractive than the Frankensteinesque Oppenheimer and Thomas splints, the Robinson splint still looks like an overstuffed gardening glove, again, a “look” not likely to cause many patients to use it.

Thus, although the medical field has a number of splints and supports that have been used for treatment of radial nerve damaged muscles, the field still lacks effective splints that have a user friendly, esthetically pleasing low-profile that, at the same time, provide for versatile, life-like operation and allow for easy interchangeability of the components that compensate for muscular and nerve deficits. Thus there has existed a long-felt need for an attractive, comfortable, and user-friendly means of splinting and treating a peripheral neurological injury to the hands and/or fingers. The need extends to a splint which the user is likely to wear, which allows the medical practitioner a wide degree of flexibility in treating the neuropathy, which is adaptable to a range of different sized hands, and is relatively inexpensive to produce.

The current invention provides just such a solution by having a low-profile, simple, orthotic glove with wrist support attachment for treating loss or impairment of extensor and/or flexor muscle function in the upper extremities, particularly in the hands and fingers, due to a peripheral neuropathy. The glove is attractive, comfortable, easy to use, inexpensive to produce, and can be used with or without the wrist support attachment. The glove has channels or tunnels into which interchangeable resilient digit extensor elements, or stays, of varying resiliency are placed such that stays of greater or lesser resiliency can be inserted if the degree of the patient's extensor muscle control changes, or the medical practitioner in charge decides that there is a need to change the support and exercise treatment regimens.

### SUMMARY OF THE INVENTION

It is a principal object of the invention to provide a glove which can be used to treat peripheral nerve damage in the upper extremities.

It is another object of the invention that the glove be attractive, easy to attach and detach, and can be worn during many daily activities such that a patient is likely to use the glove outside of the medical practitioner's office.

An additional object of the invention is that the glove can be manufactured in to cover all the patient's fingers and thumbs, be open on one or more fingers or thumbs, have removable sections which would cover one or more fingers or thumbs, be full fingered, cover only the first or second knuckle of the finger, or expose the entire finger or thumb.

It is an additional object of the invention that the glove have an optional wrist support which can be used according to the comfort of the user and the direction of the supervising medical practitioner, where the wrist support can be attached to the glove through a variety of means including snaps, zippers, Velcro®, threaded string or similar devices.

An additional object of the invention is that the glove includes a flexible, comfortable thumb strap that keeps the thumb properly positioned.

It is also an object of this invention that the glove be adjustable to a patient's hand, where such adjustability can come from one or more of the following means: the natural elasticity of the glove material, adjustable straps, adjustable zippers, adjustable snaps, Velcro®, threaded string or similar devices.

It is a further object of the invention that the glove function by keeping the patient's hand in an “open” position, such that the functional flexor muscles can close the hand to grasp objects and perform many daily, routine activities, and that the glove will then push the fingers and thumb of the hand back into the “open” position once the grasping action is no longer desired, as, for example, when a user has grasped a can, moved it to a new location, released the can, and is now ready to pick up something else.

It is another object of the invention that the glove have no outriggers, loops, wires, or other projections sticking out of it which could cause the glove to be difficult to use or unattractive to the patient.

It is an additional object of the invention that the glove allows for use by patients with less than a 25 pound grip.

It is a further object of the invention that the glove has at least one tunnel or channel in its upper surface into which one or more stays can be inserted to provide the resiliency necessary to return a finger or thumb to its “open” position after it has been closed.

An additional object of the invention is that the stays are interchangeable, such that one stay can be substituted for another easily and quickly.

It is a further objection of the invention that the tunnel or channel into which the stay is inserted can have an opening in the middle such that the stay is compressed slightly and the ends slipped into the tunnel openings before the stay is allowed to uncompress, or at either end such that the stay is slid into the tunnel and then a cover is attached over the end of the stay, securing the stay in the tunnel.

It is also an object of this invention that the stays be made in a planer, rectangular, angular, circular, hollow tube like, or multi-parted fashion.

It is a further object of the invention that the stay can be uniformly thick or tapered such that it is thicker at one end than the other.

An additional object of the invention is that the stay can be manufactured with a uniform resiliency throughout or with varying resiliency such that, for example, it has greater resiliency toward the hand and lesser resiliency at the finger portion of the glove.

It is an additional object of the invention that the stays can be manufactured with different degrees of resiliency such that stays of different resiliencies can be interchangeably inserted into the glove depending on the patient's progress and prescribed treatment regimen.

A further object of this invention is that the stays can be made of adjustable resiliency such that a stay can be adjusted over time as the patient's progress and treatment plan changes.

It is also an object of this invention that the glove be inexpensive to manufacture, and can be manufactured in a variety of sizes to accommodate different sizes of hands, wrists, and fingers.

It is another object of the invention that the glove can be manufactured from a wide variety of materials, including plastic, leather, Lycra, polyester, imitation leather, neoprene, compression garment material, and hypoallergenic materials, or perhaps breathable materials to avoid the accumulation of moisture on the palmar surface.

It is a final object of this invention that the glove and wrist support be marketable as a kit, or combination, with one of each or two of each, as would be purchased by an individual patent, or a larger number of units as would be appropriate for purchase by a hospital or medical practice.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE DRAWINGS

The following drawings form part of the present specification and are included to further demonstrate certain representative embodiments of the present invention. The invention may be better understood by reference to one or more of these drawings in combination with the detailed description of specific embodiments presented herein.

FIG. 1 is a side, perspective view of an iteration of the splint where it is a wrap-around glove, the splint which highlights the low profile of the splint along with its method of application to a human wrist.

FIG. 2 is a bottom, perspective view of an iteration of the splint where it is a wrap-around glove, showing the method of attaching the splint to the thumb, palm, and digits.

FIG. 3 is a bottom view of the splint illustrated in FIGS. 1 and 2, where the splint is not attached to a hand. The figure show a hook and loop type fastener, which is just one of any number of fasteners known in the art and which can be used for such purposes.

FIG. 4 is a front view of a stay, or resilient digit extensor element, as removed from the channel on a splint.

FIG. 5 shows a top view of the splint illustrated in FIGS. 1, 2 and 3, where the splint is not attached to a hand. In the iteration shown in FIG. 5, the glove includes one channel or tunnel for each digit positioned on the dorsal metacarpal element. In the drawing, one stay or resilient digit extensor element is shown inserted into the right-most channel on the splint.

FIG. 6 is a bottom view of another iteration of the invention in which a glove is taught, including a palmar section and a dorsal section, with the two sections stitched or otherwise joined at the seams. This figure illustrates the wrist receptacle on the palm of the glove.

FIG. 7 is a bottom view of the same glove as illustrated by FIG. 6, but with a wrist support element added to the wrist with a wrist stay inserted into the wrist receptacle.

FIG. 8 is a bottom view of the same glove as illustrated in FIG. 6, showing the wrist support and glove in unattached form, showing how the wrist support is constructed of a reinforced base with a stay receptacle, with elastic bands which can be wrapped around the wrist and secured with hook/loop or some other means of attachment.

FIG. 9 is a top view of a glove in which the stays are retained in their tunnels or channels by and retaining band which is stitched across the lower end of the stays such that stays can be added to or removed from the glove only by forcibly pulling back on the retaining band.

FIG. 10 is a top view of the glove of FIG. 9, with the retaining bank pulled back to reveal the bottom ends of the stays.

FIG. 11 is a front view of a stay.

FIG. 12 is a front view of a wrist support.

FIG. 13 is a side view of a wrist stay.

FIG. 14 is a top view of a wrist stay.

FIG. 15 is a bottom view of a wrist support

It is intended that the drawings of the invention described above are non-limiting and that other features and advantages of the invention will be apparent from the brief description of the drawings, the drawings themselves, the detailed description of the invention, and the appended claims.

### DETAILED DESCRIPTION OF THE EMBODIMENTS

The present invention concerns nerve splints for the hand that comprise an orthotic glove. These splints are useful in treating patients having a peripheral neuropathy that results in permanent or temporary full or partial loss of extensor muscle control of one or more digits of a hand. The glove portion of a splint according to the invention provides for customized, patient-specific treatments by including a resilient digit extensor element contained or otherwise disposed in a channel, pouch, or other structure positioned on the dorsal portion of at least a digit section the glove. The extensor element provides sufficient resilience to return the digit to an extended position relaxation of the flexor muscle for that digit. As the amount of tension required to return a particular digit to an extended position will vary between digits and patients, and will even vary over time for the affected digit(s) of a particular patient during the healing process, in preferred embodiments the gloves provide for easy relate to the resilient digit extensor elements. Other features of the splints of the invention relate to their low-profile and aesthetically pleasing nature. Because of their low profile, the splints of the invention may be worn at all times, even while dressing and sleeping.

Turning to the actual shape of the invention, there are several iterations of the invention, but all iterations basically comprise a glove in which the material covering the fingers and thumb on the dorsal and palmer areas of the hand extend over part or all of the digits. In some iterations, one or more of the finger covers are open at the distal end, such that the finger tips of a wearer are exposed when the glove is worm. Such openings can be manufactured to provide exposure of any desired portion of one or more fingers. In a preferred embodiment, the glove material that contacts the skin of the hand and fingers is made of a pliable and resilient material such as cloth, cotton, plastic, leather, or a synthetic material such as neoprene, nylon, or a hypoallergenic material. In certain circumstances where the wearer has extreme sensitivity to touch, the surface of surface of the glove that contacts the skin can comprise fur, cotton, or another soft material.

A key component to the invention is the capability of the glove to accept a stay or resilient digit extensor element, into a tunnel or channel design and manufactured into the dorsal side of the glove. The channel or tunnel substantially tracks that portion of the glove intended for the insertion of a finger. The channel, or in a preferred embodiment, channels—at least one for each finger—are made from a second layer of a material attached or otherwise affixed to the glove material. The second material may be different or the same as the material in contact with the skin. The tunnel or channel material is attached to the dorsal side of the glove such that one or more tunnels or channels are formed along the dorsal portion of the glove along the back of part of one or more fingers, and, optionally, the thumb. Preferably, the tunnels along the back of the one or more fingers extend the entire length of the finger portions and are sealed with respect to the first material layer on three sides, (i.e., at the leading or distal edges of the finger portions and along each side of the pocket extending from said leading edge to an area near where the wearers knuckles would rest in the glove, where the tunnel openings are formed). Thus, preferably, the glove has at least four such tunnels and, optionally, five such tunnels, including one for the thumb, along the upper back edge (i.e., along the proximal portion of the wrist) of the glove, extending along the back side of each digit for inserting resilient extensor strips.

The channels are designed and manufactured for insertion and removal of resilient digit extensor strips, or stays, that compensate for loss of digit extensor function. In preferred embodiments, the gloves allow for removal and replacement of the resilient digit extensor elements as the needs of the patient change over time. The resilient digit extensor elements are typically elongate strips made of a synthetic material having memory or spring-like qualities. In some embodiments, the resilient digit extensor elements are planar and linear and of a length specific for the size of the glove and the channel into which the element is e inserted or otherwise positioned in the glove. In preferred embodiments in which the gloves are configured to accept two or more resilient digit extensor elements, the resilient digit extensor elements used for a particular have different degrees of resilience matched to the strength or weakness of finger muscles of the wearer.

The glove works by providing a mechanisms and means for applying adjustable and controlled amounts of extensor tension to the patient's fingers so that an appropriate level of resistance can be set for an individual patient, thereby allowing the wearer to realize full flexor capability while keeping the fingers in a neutral, relaxed position when not flexed in an extended an relaxed posture. Another iteration of the invention concerns a larger orthotic glove into which a patient's hand fits (as opposed to the “wrap around” version which uses straps to attach the patient's fingers, palm and wrist to the device) operably connected to wrist support that provides for wrist extension.

The invention, in all its iterations, provides a device and method for the treatment of radial and peripheral nerve injuries. The invention provides methods of compensating for full or partial, permanent or temporary loss of extensor function of one or more digits of a patient suffering from a radial or peripheral neuropathy. In addition to protecting the injured hand, the invention allows for a medical practitioner to customize treatments for individual patients by selecting particular resilient digit extensor elements for each affected digit. Also, over time and as a patient's condition improves or worsens, some or all of the various resilient digit extensor elements may be exchanged for other resilient digit extensor elements having differing levels of resilience depending upon the success or failure of the wearer to increase voluntary control of the flexor and/or extensor muscles for each individual digit.

The figures, while not limiting the invention to any one or several particular iterations, are design to provide an illustrative view of several of the most promising iterations.

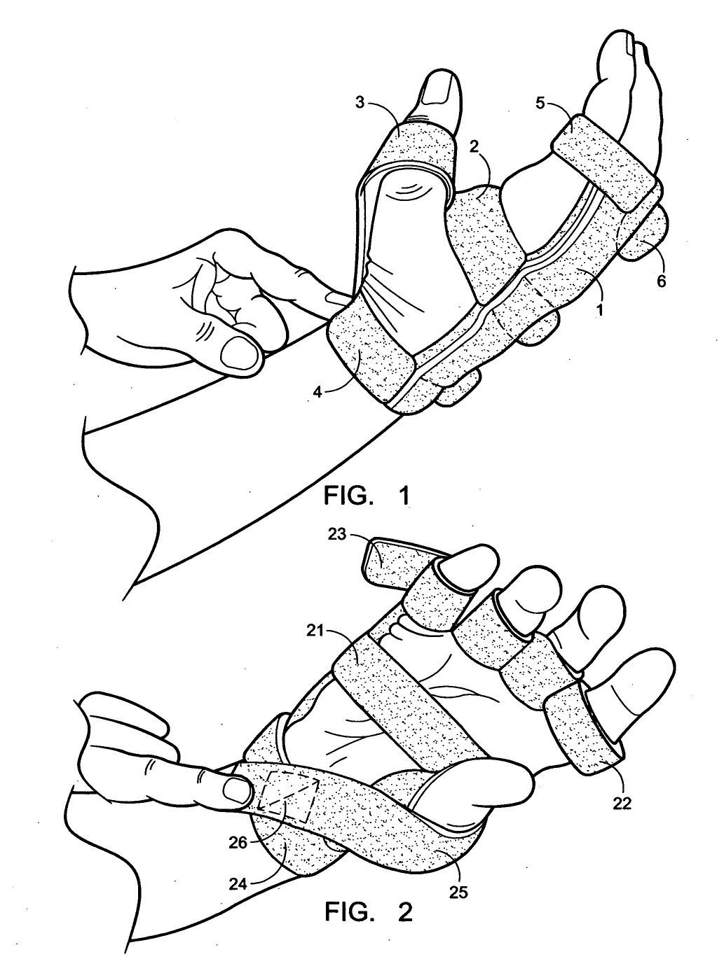

FIG. 1 is a side, perspective view of an iteration of the splint where it is a wrap-around glove, the splint which highlights the low profile of the splint along with its method of application to a human wrist. The glove has a dorsal side (1) which fits over the dorsal metacarpal portion of a user's hand. Rather than having a solid bottom or palmar side, as will be seen in additional iterations of the invention, this iteration has straps that wrap around the users had to secure the dorsal side (1) to the user's hand and wrist. There is a palmar retention strap (2) which secures the glove to the hand, a thumb retention strap that secure the glove to the thumb, a wrist retention strap (4) that secures the glove to the wrist, and digital retention straps (5) that restrain the fingers against the stays (not shown in this figure). Each retention strap has a means of wrapping the retention strap around the desired object and securing the retention strap. In this figure, the digital retention strap (5) is wrapped around a finger, and attached to its backside (6) with Velcro®, snaps, or other means of attachment. As such, each retention strap, whether its purpose is mainly to secure the glove to the hand, as in the palmar retention strap (2), or to position the body for treatment and functional purposes, such as the digital retention straps (5), can be adjusted so that the glove fits a wide range of hand sizes and shapes, and so that a user can customize the fit for the user's comfort. It is envisioned that this iteration, along with the others described in this application, can be made in a wide variety of colors, including skin colors such that the glove will blend in with a person's skin and not be obvious.

FIG. 2 is a bottom, perspective view of an iteration of the splint where it is a wrap-around glove, showing the method of attaching the splint to the thumb, palm, and digits. The palmer retention strap (21) is a band of material, preferably a thin yet sturdy material, with, optionally, a degree of elasticity. Over each finger there is a digital retention strap (22), which is wrapped around the finger and then secured by taking the distal end (23) of the digital retention strap (22) and securing it to the back of the digital retention strap (22) through Velcro®, snaps, or other means of attachment. The wrist retention strap (24) serves to anchor the glove to the wrist, thereby avoiding the possibility that the glove would “run up” the user's hand. The thumb retention strap (25, wraps around the user's thumb and then is pulled down and laterally across the bottom side of the user's thumb, and secured to the outer surface of the wrist retention strap (24) on a portion of the wrist retention strap (24) that has a section of Velcro®, snaps, or other means of attachment on a location on the wrist retention strap (24) that is optimally located to receive a mating section of Velcro®, snaps, or other means of attachment on the distal end (26) of the thumb retention strap. As can be seen from FIGS. 1 and 2, with the three points of retention, wrist, palm and fingers, a stay put in the backside of the glove will hold “open” the fingers, as is necessary for not only the protection and treatment of damage to the peripheral or radial nerves, but also to allow the user to effectively grip objects and have his/her hand move back to an “open” position from which he/she can grip additional objects once the user releases the first object. FIGS. 1 and 2 show the user's hand in an “open position”.

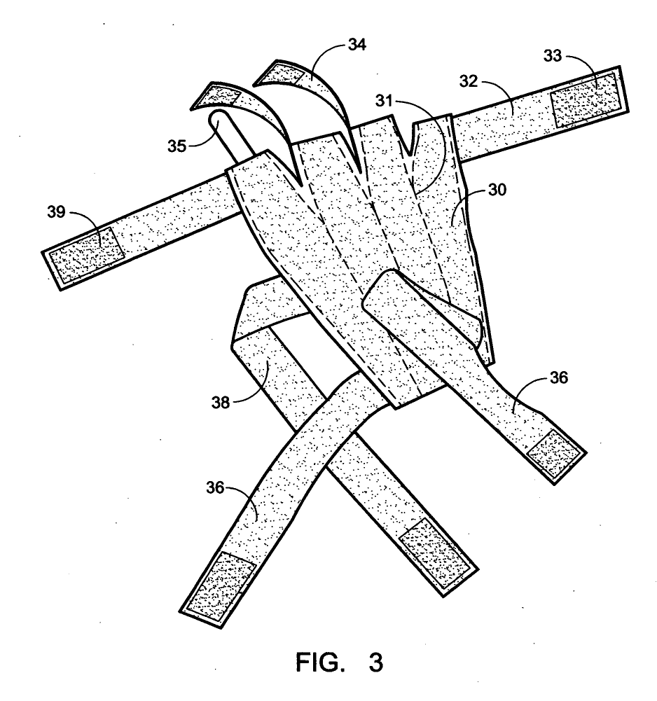

FIG. 3 is a bottom view of the splint illustrated in FIGS. 1 and 2, where the splint is not attached to a hand. The figure show a hook and loop type fastener, which is just one of any number of fasteners known in the art and which can be used for such purposes. The glove illustrated here is the “wrap around” iteration, so there is only one flat piece of material making up the dorsal side, of the bottom (30) of the dorsal side is shown in this figure. The dorsal side is made up of four finger sections, each stitched together or attached to each other by means other than stitching at seam lines (31). It is also envisioned that the dorsal side can be made from one solid piece of material. Coming off the digital extensions of each finger section is a digital retention strap (32) which has on its distal end (33) and (39) a piece of Velcroo, snaps, or other means of attachment, which then wraps around the finger, as illustrated by (34) and is secured to another piece of Velcro®, snaps, or other means of attachment to which it mates on the backside of the digital retention strap. A stay (35) or resilient digit extensor element is inserted into a channel or tunnel (not shown in this figure), and secured through an optional variety of means (not shown in this figure). The stay provides the retention for the fingers, such that a person with radial or peripheral nerve damage who can clench his/her hand but not unclench it can grasp an object, release it, and have the stays return the hand to an “open position”. The glove also has, in this iteration, two wrist retention straps (36) which wrap around the wrist and can be secured to each other. The glove also has a thumb retention strap (38) which wraps around a thumb and can be secured to the outer surface of the wrist retention strap (36). It should be noted that the glove shown in FIG. 3 is a left-handed glove, but all iterations of the invention can be made in right- or left-handed versions. It is also envisioned that this “wrap-around” version can be made in a universal model, where it can be merely flipped over for use by use on either the right hand or the left hand, whichever is injured.

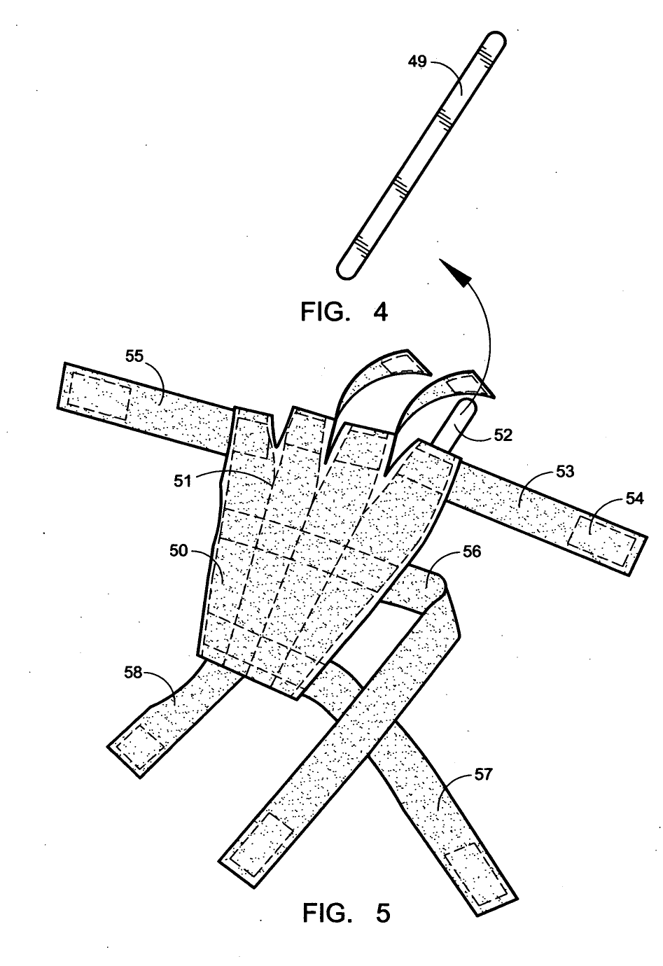

FIG. 4 is a front view of a stay, or resilient digit extensor element, as removed from the channel on a splint. The stay (49) in this figure is a “popsicle stick” iteration, where the stay is of uniform width and thickness throughout its length, with rounded edges for safety and ease of insertion into the channel or tunnel (not shown in this figure). It is also envisioned that stays can be tapered in both width and thickness, of different shapes, of different resiliencies, and even of different resiliencies within the same stay, as is needed to treat different injuries over the progression of the treatment. The digit extensor elements can be constructed of any resilient material having memory and/or spring-like qualities, resilience may be engineered using a synthetic carbon-based plastic, rubber, polyethylene, polycarbonate, polyvinyl chloride, or metal or metal alloy or a composite of all of the above. In some embodiments, the strips are planar, elongate, and linear and of a length specific for the size of the glove and the particular digit section with which it is to be associated.

Depending on the configuration of a particular splint, the resilient digit extensor elements 51 may be removable, or they may be sealed into a channel disposed on the dorsal side of the glove in alignment with a digit section. In other iterations, the resilient digit extensor elements are custom made with respect to their degree of resilience, thus making available a number of different extensor elements having different degrees of resilience, in addition to different lengths, widths, etc. As a result, splints customized to particular patients can be produced, according to the particular disability then suffered by a patient with nerve damage. In operation, preferred embodiments of the splints of the invention are adjustable in terms of the amount of extensor tension employed with respect to each digit in need of external extensor function (i.e., to compensate for nerve and/or muscle damage associated with the particular digit) so that an appropriate level of resistance can be set for a particular patient at a given time. Such splints allow the user to realize improved (and in some cases, up to and including full) extensor function, which can also promote healthy, full range-of-motion flexor function. As will be appreciated, the splints of the invention maintain the digit(s) being treated in an extended, relaxed posture in the absence of muscle flexion. In some embodiments, adjusting tension is performed by simply removing the particular resilient digit extensor element(s) to be changed from the pockets in the glove and inserting a different digit extensor element having a higher or lower degree of resilience. The degree of resilience can be engineered into the extensor elements not only by the type of material used, but also by the thickness and/or pre-selected curvature of the extensor elements. For example, a resilient digit extensor element may be produced with a thickness having a particular stiffness that can be calibrated based on known standards of flexibility, such as where the degree of resilience can be measured in terms foot-pounds.

As mentioned, one iteration of the stay is directed toward a stay with a tapered appearance, which has varying amounts of resilience throughout its length. This provides an obvious advantage in that the tension directed to finger muscles of the distal phalanx bone sections to have a greater or lesser degree of resilience than the metacarpal section muscles, depending upon the orientation in which the extensor elements are placed in the glove. For example, if the thin section is placed in the digit-side pocket, less force will be available to counter flexor muscle tension than if the extensor element was installed in the reverse orientation in the glove.

Additionally, the resilient digit extensor elements can be constructed with a slight curvature instead of being substantially flat or planar when not opposed by another force (e.g., flexor muscle tension). For example, a curved extensor element can be placed into a pocket overlying an affected digit so that the curvature of the inserted extensor element causes the splint to cause the digit to be rest in a slightly flexed position (i.e., as if the flexor muscle controlling the digit contracted to exert a slight tension on the digit). Alternatively, an extensor element can be curved upwardly to that upon insertion into the pocket the resting curvature of the extensor element flexes the digit in a slightly hyper-extended position. This spring/memory quality of the extensor elements allows for use of the splint for patients experiencing loss of extension and/or flexor muscle capability alike.

FIG. 5 shows a top view of the splint illustrated in FIGS. 1, 2 and 3, where the splint is not attached to a hand. In the iteration shown in FIG. 5, the glove includes one channel or tunnel (50) for each digit positioned on the dorsal metacarpal element. In the drawing, one stay (52) or resilient digit extensor element is shown inserted into the right-most channel on the splint. The glove has a line of stitching (51) which separates each tunnel or channel (52) from the others. A stay (52) of differing resiliency, shape, thickness and other characteristics can be inserted into each tunnel (50), thereby allowing the medical practitioner to adjust and modify the treatment regimen as the patient progresses. For each finger there is a digital restraining strap (52) and (55), with an adhesive means (54) for attaching the digital restraining strap after it is wrapped around the finger. The glove also has a palmar retaining strap (56) which is stitched or attached by other means to the dorsal surface of the glove, and a thumb restraining strap (57), along with a wrist restraining strap (58).

FIG. 6 is a bottom view of another iteration of the invention in which a glove is taught, including a palmar section (60) and a dorsal section (not shown in this figure), with the two sections stitched or otherwise joined at the seams. This figure illustrates the wrist stay receptacle (63) on the palm of the glove. The glove has digital retaining straps (64), a thumb retaining strap (61), which has a means of attachment (62) which attaches to the surface of the wrist stay receptacle (63). There is also a wrist retaining strap (65) which is used to secure the glove to the wrist of the user such that the glove does not “ride up” or fall off the user's hand. The wrist stay receptacle (63) serves as a receptacle for the wrist support element (not shown in this figure), which is an object that connects the wrist splint (not shown in this figure) to the glove.

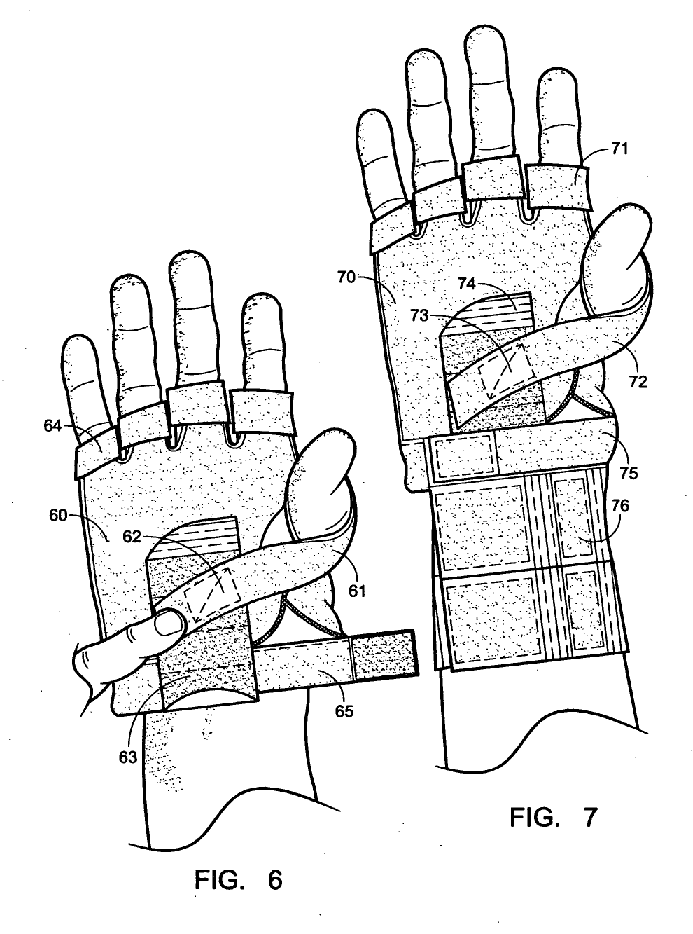

FIG. 7 is a bottom view of the same glove as illustrated by FIG. 6, but with a wrist support element (76) added to the wrist with a wrist stay (not visible in this figure) inserted into the wrist receptacle (74). Here, the glove (70) has digital retaining straps (71), a wrist retaining strap where in this case serves to not only secure the glove to the user's wrist, but also to cover the connection between the wrist support element (76) and the glove (70), thereby adding to the security of the connect and ensuring that the wrist stay (not shown in this figure) will remain secure in the wrist stay receptacle (74). The thumb retaining strap (72) has an adhesive means (73) by which the thumb retaining strap (72) is attached to the outside of the wrist stay receptacle (74). By adding the wrist support element (76) to the glove (70), the user is given additional support for his/her hand and wrist, and is particularly applicable to users who have experienced nerve damage and impairs their ability to maintain wrist positions as well as undenching their hand.

FIG. 8 is a bottom view of the same glove as illustrated in FIG. 6, showing the wrist support and glove (80) in unattached form, showing how the wrist support is constructed of a reinforced base with a wrist stay base channel (85), with bands (87), preferably made of a material with some elasticity, which can be wrapped around the wrist and secured with hook/loop or some other means of attachment. A wrist stay (86) can be inserted into the wrist stay base channel (85), such that an appropriate amount of the wrist stay (86) is available for insertion into the wrist stay receptacle (84) on the glove (80). The wrist retaining strap (83) of the glove (80) then can be wrapped around the junction between the wrist stay receptacle (84) and the wrist stay base channel (85), thereby further securing the connection between the two units. The glove in this iteration also has digital restraining straps (81) and a thumb restraining strap (82). The glove illustrated by this figure is a right-hand glove, but it is envisioned that left-handed gloves could also be made, and that even a glove with wrist stay receptacles and stays on either side could be manufactured such that one glove could be worn on either the right or left hand. This iteration is also designed so that a user can use either the glove or the wrist support element optionally, depending on the treatment regimen and injury.

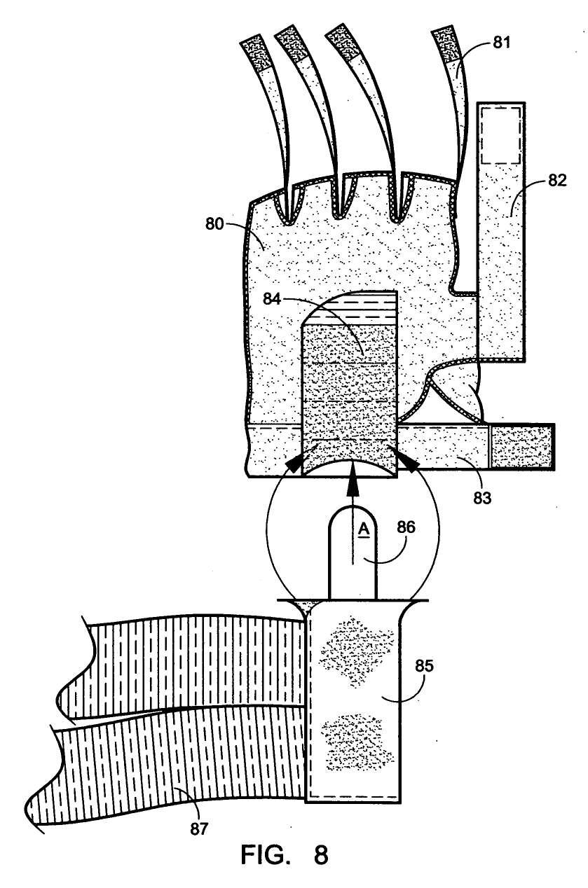

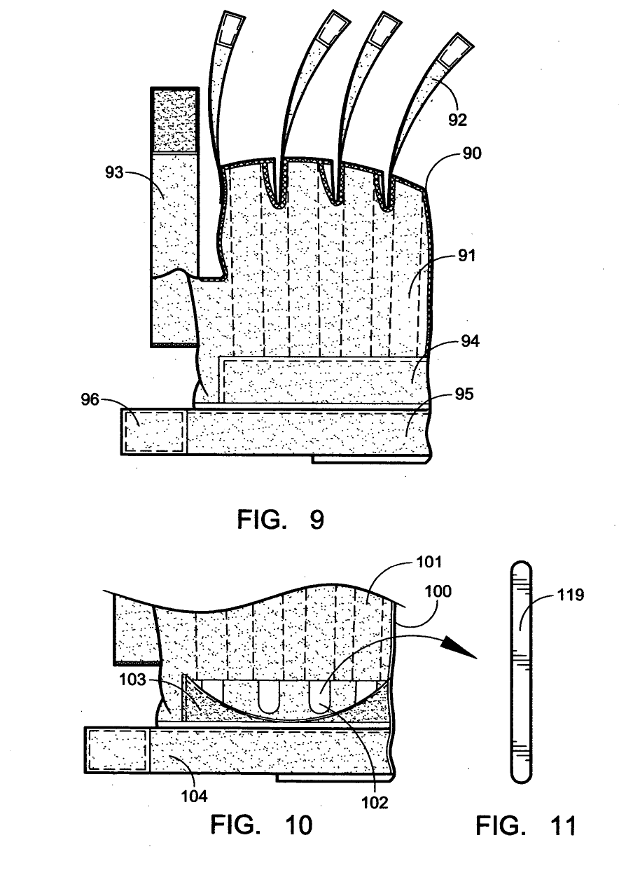

FIG. 9 is a top view of a glove (90) in which the stays (not visible in this figure) are retained in their tunnels (91) or channels by and a retaining band (94) which is stitched across the lower end of the stays such that stays can be added to or removed from the glove only by forcibly pulling back on the retaining band. This iteration of the glove, which can be made in both “full glove” and “wrap around” versions, has digital retaining straps (92), a thumb retaining strap (93), a wrist retaining strap (95) which a means of attachment (96). The key feature of this version, however, is the retaining band (94). The tunnel (91) or channel is sewn or otherwise attached or sealed at the digital end, such that the tunnel (91) is has only one end open, that being the end closest to the user's wrist. Stays can be slid into the channel (91) and the edge of the retaining band (94) is pulled back to allow such insertion, then allowed to fall back into place securing the stay in its channel.

FIG. 10 is a top view of the glove of FIG. 9, with the retaining band (103) pulled back to reveal the bottom ends (102) of the stays. The stay has already been inserted into the channel (101), and the retaining band (103) has been pulled back to allow access to each stay. After the stay has been inserted or removed, the retaining band (103) is allowed to return back to its normal position, as illustrated by (94) in FIG. 9. It is envisioned that the retaining band (103) can be made from a thin but sturdy material, with, optionally, some flexibility which will allow it to snap back into place over the bottom ends (102) of the stays.

FIG. 11 is a front view of a stay. The stay (119) is of the uniformly wide and thick “popsicle stick” variety, but it is envisioned that the stays can be designed and manufactured in a wide variety of shapes, including those tapered in both width and thickness, of different shapes, of different resiliencies, and even of different resiliencies within the same stay or of adjustable resiliencies within the same stay, as is needed to treat different injuries over the progression of the treatment.

FIG. 12 is a front view of a wrist support (120). The invention has a wrist stay base channel (121) which is a channel defined by two sides of material which are stitched or otherwise attached to each other on their two long sides and the bottom end, and left open at the top end. A wrist stay (122) can be partially inserted into the wrist stay base channel (121) so that an appropriate length of the wrist stay (121) extends out of the opening of the wrist stay base channel (121) for insertion into the wrist stay receptacle (not shown) of the glove (not shown). To attach the wrist support (120) to the wrist is one or more lengths or bands (123) of a flexible material, optionally with some elasticity. In this figure there are two such lengths.

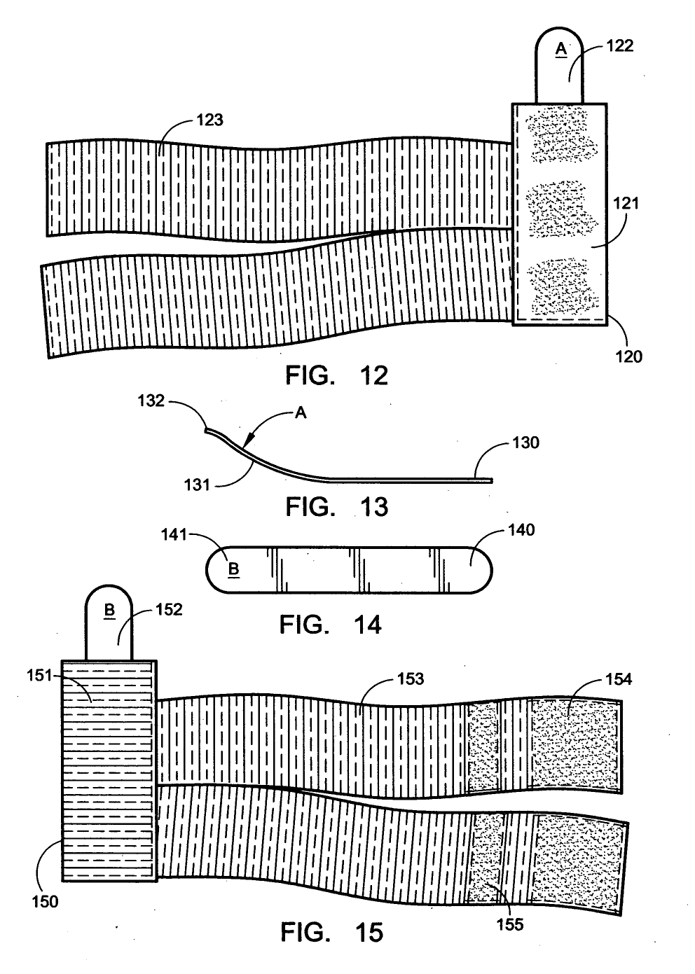

FIG. 13 is a side view of a wrist stay, showing that the bottom end (130) that is designed to fit into the wrist stay base channel (not shown in this figure) is preferably flat and linear, but the portion of the wrist stay that enters the wrist stay receptacle in the glove (not shown) has a bend (131) which is designed to provide an ergonomic fit with a user's palm, and a tapered section (132) at the tip which is designed to prevent the wrist stay from digging into the palm of the user and causing discomfort.

FIG. 14 is a top view of a wrist stay, showing the “popsicle stick” iteration of the wrist stay, where the wrist end (140) and the palm end (141) look identical from a flat, front view.

FIG. 15 is a bottom view of a wrist support (150). The invention has a wrist stay base channel (151) which is a channel defined by two sides of material which are stitched or otherwise attached to each other on their two long sides and the bottom end, and left open at the top end. A wrist stay (152) can be partially inserted into the wrist stay base channel (151) so that an appropriate length of the wrist stay (152) extends out of the opening of the wrist stay base channel (151) for insertion into the wrist stay receptacle (not shown) of the glove (not shown). To attach the wrist support (150) to the wrist is one or more lengths or bands (153) of a flexible material, optionally with some elasticity. In this figure there are two such lengths. This figure shows two areas of attachment, 154 and 155, which in this iteration are hook and loop means of attachment, which will wrap around the wrist and attach to mating surfaces on the backside of the bands (153). While this figure shows hook and loop means of attachment, it is also contemplated that means of attachment could also include a series of snaps on the underside of the bands (153) which could be adjustably connected to a mating series of snaps on the backside of the bands (153).

As those in the art will appreciate, the hand splints of the invention have a substantially flat or “low” profile due to the thin nature of the materials and components used in the glove portion. The low profile enhances the aesthetic quality of the glove, in that the splint is much less visible to others when worn, and in any event appears very much like conventional weight-lifting, sailing, or other sports gloves or fashion accessories. In addition, the low profile nature of the splint allows the wearer to easily wear the glove while conducting normal or routine activities such as in dressing, bathing, and eating.

Further still, in yet another embodiment, the invention provides for a treatment regimen wherein one or more of a first set of extensor elements are exchanged over time with extensor elements having a greater or lesser the degree of resilience and/or curvature depending upon the progress being attained with the then-current one or more resilient digit extensor elements.

In still other embodiments, the splint can be used to treat various neuropathy disorders, including those that affect both the ability to extend the digits and the ability to flex the digits. Similarly, the splints of the splints of the invention can also be used by healthy subjects, for example, to increase grip strength (by overcoming resistance to flexion of digit flexor muscle). An embodiment useful for therapy is described in the following

### EXAMPLE

### Example 1

Where a patient has suffered a nerve or muscle injury that affects the ability of the patient to extend at least one digit of a hand, a glove according to the invention may be placed on the hand to effect treatment. Preferably the patient is first tested by his/her attending physician, physical therapist, or other rehabilitation provider to determine the extent of the nerve and/or muscle deficit with respect to the digit(s) involved. Depending upon the ability or inability to extend or flex the at least one digit, a resilient digit extensor element of appropriate resilience and design is inserted between the pockets of the glove that are designed to effect therapy for the particular digit. The patient is then instructed to wear the splint as necessary or according to an exercise/treatment protocol.

If, during the course of therapy, the patient achieves sufficient progress (or not), a determination may be made to exchange one or more of the resilient digit extensor elements for others have differing degrees of resilience in order to further advance therapy.

As will be apparent to those of ordinary skill in the art in the light of the foregoing disclosure, many alterations and modification are possible in the practice of this invention without departing from the spirit or scope thereof. Accordingly, the scope of the invention is to be constructed in accordance with the substance defined by the following claims.

The invention illustratively described herein suitably may be practiced in the absence of any element(s) not specifically disclosed herein. Thus, for example, in each instance herein any of the terms “comprising consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

What I claim as my invention is:

1. An orthotic glove for treating radial or peripheral nerve damage, comprising:
a dorsal metacarpal element, where the dorsal metacarpal element covers at least a portion of a user's hand,
a ventral palmar element, where the ventral palmar element covers at least a portion of a user's hand,
a flexible thumb strap that keeps the thumb properly positioned, where the thumb strap is easy to secure and comfortable to the user,
a means of attachment by which the dorsal metacarpal element and the ventral palmer element are attached to each other,
at least one digit section extending from the dorsal metacarpal element and palmar element past the MP or Metacarpophalangeal joint,
at least one channel capable of accepting a resilient digit extensor element, where each channel spans from the dorsal metacarpal element to a dorsal area of a digit section, and where each channel has a means of retaining the digit extensor element within it, such that the stays are interchangeable, such that one stay can be substituted for another easily and quickly,
at least one resilient digit extensor element in a channel, and,
where, the glove functions by keeping the patient's hand in an “open” position, such that the flexor muscles can close the hand to grasp objects and perform many daily, routine activities, and that the resilient digit extensors of the glove will then push the fingers and thumb of the hand back into the “open” position once the grasping action is no longer desired, and,
optionally, a wrist support device, where the wrist support device comprises a wrist stay base channel, a wrist stay of a length such that the wrist stay can be partially inserted into the wrist stay base channel so that an appropriate length of the wrist stay extends out of the opening of the wrist stay base channel and can be inserted into a wrist stay receptacle of the glove, and, one or more lengths or bands, consisting of a length of flexible material, optionally with some elasticity and means of adjustment and attachment to allow a user to snugly and comfortably fit the writ support device to his/her wrist, where, by inserting the wrist stay into the wrist stay receptacle on the glove, the user can operably connect the wrist stay and the glove.

2. The glove of claim 1, where, the glove additionally comprises:
a means of adjustment by which the glove can be adjusted to an individual user's hand size, where the means of adjustment can be the natural elasticity of the glove material, adjustable straps, adjustable zippers, adjustable snaps, Velcro®, threaded string or similar devices, and,
a means of securing the glove to the user's hand, where the glove is designed such that it can be easily and conveniently put on and taken off, and,
where, the glove can be made in a variety of sizes to accommodate the different sizes of hands,
where, the glove can be manufactured from a material, selected from a group comprising: plastic, leather, lycra, polyester, imitation leather, neoprene, and compression garment material,
where the glove can be made for the right hand or the left hand, and,
where the glove is of a low profile such that no portion of the glove extends above the palm or dorsal portions of the hand by more than ½ inch, such that it can be worn during many daily activities such that a patient is likely to use the glove outside of the medical practitioner's office,
where the material from which the glove is made can, optionally, be of a skin color or other color designed to make the glove unobvious when being worm, and,
where the glove has no outriggers, loops, wires, or other projections sticking out of it more than ¼ inch above the surface of the glove,

3. The glove of claim 2, where the glove additionally comprises at least one digit section that extends toward the end of a user's finger to at least the Proximal interphalangeal joint (PIP joint).

4. The glove of claim 2, additionally comprising a separate channel for each of a plurality of digit sections and a resilient digit extensor element which is disposed in each channel, where each resilient digit extensor element has an independently selected resilience such that a user or medical practitioner can select digit extensor elements of different resistances for different fingers depending on different neural and muscle strengths and different treatment regimes, and can exchange individual resilient digit extensor elements as a patient's treatment progresses and the patient's condition changes.

5. The glove of claim 1, where the digit section extends toward the end of a user's finger to at least the Distal interphalangeal joint.

6. The glove of claim 1, where the digit section extends such that it covers the entire finger.

7. The channel of claim 1, where the channel into which the resilient digit extensor element is inserted has an opening in the middle such that the resilient digit extensor element can be compressed slightly and the ends of the resilient digit extensor element slipped into one or more tunnel openings, before the stay is allowed to uncompress and flatten out into the tunnel, thereby securing itself into the tunnel.

8. The channel of claim 1, where the channel has a dosed end, a covered mid-section, and an open end, where, the resilient digit extensor element is slid into the open end of the channel and then a cover is attached over the end of the resilient digit extensor element and the open end of the channel, thereby securing the resilient digit extensor element in the channel.

9. The resilient digit extensor element of claim 1, where the resilient digit extensor element comprises a resilient material from the group consisting of a synthetic carbon-based plastic, rubber, polyethylene, polycarbonate, polyvinyl chloride, a metal, a metal alloy, and a combination of any two or more of the foregoing materials.

10. The resilient digit extensor element of claim 1, where the resilient digit extensor element can be made in planer, rectangular, angular, circular, hollow tube-like, or multi-parted designs.

11. The resilient digit extensor element of claim 1, where the resilient digit extensor element can be manufactured with varying degrees of resiliency within the same resilient digit extensor element.

12. The resilient digit extensor element of claim 1, where wherein said resilient digit extensor element is curved.

13. The resilient digit extensor element of claim 1, where said resilient digit extensor element has two ends and is tapered such that one end is thicker than the other end.

14. The resilient digit extensor element of claim 13, where said resilient digit extensor element has less resilience at a thin end than at a thick end.

15. The resilient digit extensor element of claim 1, where the individual resilient digit extensor element can be adjusted over time as the patient's progress and treatment plan changes.

16. The glove of claim 1, where the glove allows for use by patients with less than a 15 pound grip.

17. The glove of claim 1, where the glove allows for use by patients with less than a 25 pound grip.

18. A retail package, comprising at least one orthotic glove according to claim 1.

19. The retail package of claim 21, where the retail package comprises at least one other glove, wherein at least one of the gloves is configured for wearing on a right hand and at least one of the gloves is configured for wearing on a left hand, and, optionally, at least one wrist support.

20. A method for treatment of a full or partial loss of extensor function for one or more digits of a hand, the method comprising placing an orthotic glove on a hand of a subject who suffers from full or partial loss of extensor function for one or more digits of the hand, thereby compensating for full or partial loss of extensor function for one or more digits of the hand, where such glove comprises:
a dorsal metacarpal element, where the dorsal metacarpal element covers at least a portion of a user's hand,
a ventral palmar element, where the ventral palmar element covers at least a portion of a user's hand,
a means of attachment by which the dorsal metacarpal element and the ventral palmer element are attached to each other,
a means of adjustment by which the glove can be adjusted to an individual user's hand size,
at least one digit section extending from the dorsal metacarpal element and palmar element such that the digit section covers at least to the first PIP joint,
at least one channel capable of accepting a resilient digit extensor element, where each channel spans from the dorsal metacarpal element to a dorsal area of a digit section,
and where each channel has a means of retaining the digit extensor element within it, where the glove can be made for the right hand or the left hand,
at least one resilient digit extensor element in a channel, and,
optionally, a wrist support device, where the wrist support device can be connected to the glove to provide support for the wrist.
